(19) United States
(12) Patent Application Publication
(10) Pub. No.: US 2019/0167743 A9
(43) Pub. Date: Jun. 6, 2019

(54) COMPOSITION AND METHODS OF USE FOR IMMUNE SYSTEM SUPPORT

(71) Applicant: Sarath Malepati, Los Angeles, CA (US)

(72) Inventor: Sarath Malepati, Los Angeles, CA (US)

(21) Appl. No.: 15/266,946

(22) Filed: Sep. 15, 2016

Related U.S. Application Data
(60) Provisional application No. 62/219,013, filed on Sep. 15, 2015.

(51) Int. Cl. A61K 36/28; A61K 9/00; A61K 9/48; A61K 33/30; A61K 31/375

(52) U.S. Cl. CPC A61K 36/28; A61K 9/0053; A61K 9/48; A61K 9/4875; A61K 31/375; A61K 9/4866; A61K 33/30

(57) ABSTRACT

An oral composition of Echinacea, vitamin C, and zinc in a nanoparticle delivery system and dose schedule method of consumption that supports the immune system while reducing inappropriate antibiotic use. In a preferred embodiment, a single dose is composed of one Echinacea capsule and one zinc-vitamin C capsule with the particle contents contained within a liposome that enhances enteric gut absorption and delivery, which can also include use of nanoparticles and honey-based materials. In a preferred embodiment, doses are scheduled with higher frequency at the initial onset of symptoms and subsequently tapered off over 5 days.

### RELATED U.S. APPLICATION DATA

Provisional Application No. 62/219,013, filed Sep. 15, 2015.

### FIELD OF INVENTION

The field of this invention relates generally to over-the-counter medications and supplements for human consumption, and their method of use, intended to improve health, support the immune system, and avoid or shorten viral illnesses such as the common cold and other upper respiratory infections. More specifically, this invention comprises a composition of materials, including Echinacea, vitamin C, and zinc, in a targeted delivery system packaged in a preferred dose and dose schedule that optimizes their effectiveness, and may include honey and a nanoparticle delivery system and method so as to further increase the efficacy of the treatment.

### BACKGROUND

A significant global public health problem today is the overuse and inappropriate use of antibiotics. Antibiotics are medicines designed primarily to treat and in some cases, prevent, bacterial illnesses. A recent 2013 retrospective study conducted by the Centers for Disease Control found that in the year 2010, U.S. physicians and associated health care providers prescribed 258 million courses of antibiotics for a population of approximately 309 million Americans. As described in Hicks L. A., Taylor T. H., Hunkier R. J. (2013). U.S.

Outpatient Antibiotic Prescribing, 2010. New England Journal of Medicine, 368, 1461-62, this translates, on average, to 833 antibiotic prescriptions for every 1000 people.

As reported by Cadieux G., Tamblyn R., Dauphinee D., Libman M. (2007). Predictors of inappropriate antibiotic prescribing among primary care physicians. Canadian Medical Association Journal, 177(8), 877-83, the most common reason that patients are prescribed antibiotics in the primary care setting is for upper respiratory infections (URIs). It has been reported by Adams P. F., Hendershot G. E., Marano M. A. Centers for Disease Control and Prevention/National Center for Health Statistics (1999), and according to current estimates from the National Health Interview Survey, 1996 (reported in Vital Health Statistics, 10, 200, 1-203), that URIs are the most common infectious disease among humans, yet greater than 75% of URIs are not in fact caused by bacteria, but rather viruses. Antibiotics have no effect on viruses. Rapid diagnostic tests assessing whether an infection is bacterial or not are generally limited to very specific bacterial strains and can be limited by their accuracy as well as user error. Formal bacterial culture via nasal or throat swab to assess bacterial growth takes 48-72 hours for the results. By the time a throat culture can be finalized, a bacterial infection may spread even further with serious consequences for the patient. This presents a better-to-be-safe-than-sorry situation for the provider. Thus, the diagnostic limitations coupled with patient pressure for a tangible solution from the healthcare provider are the primary drivers of inappropriate antibiotic prescriptions for viral illnesses. In fact, according to a June 2014 WebMD/Medscape survey, 95% of licensed medical prescribers in the United States admit to writing antibiotics when unclear of their necessity. The number one cited reason for this inappropriate use of antibiotics is patient expectation.

Consequently, the great majority of people taking antibiotics for URis are receiving them unnecessarily while their actual disease is not being treated. The recent observational study in Family Practice reported by Dekker A. R. J., Verheij T. J. M., Van der Welden A. W. (2015) confirms this, and also confirms that inappropriate antibiotic prescription for respiratory tract indications is most prominent in adult patients. In this particular study, 46% of antibiotics written for respiratory tract infections were not indicated based on clinical guidelines.

Unfortunately, the over-prescription of antibiotics is not risk-free. Antibiotic misuse can have serious health implications for the individual taking them. In addition to not effectively treating the viral illness, the antibiotics can actually cause harm, for example in the form of eliminating normal bacteria that regulate critical physiological functions. The over-use of antibiotics can also lead to further health complications for the individual; as well as for the population as a whole. As another example, inappropriate antibiotic use can select for multi-drug resistant organisms, colloquially called “super bugs,” that cannot be eradicated by conventional methods. These “super bugs” pose a serious and growing worldwide health concern. Despite these dangers, many patients with URIs continue to come to their physicians and health care providers requesting antibiotics that cannot and should not be used to treat viral infections. See Dekker A. R. J., Verheij T. J. M., Van der Welden A. W. (2015), supra.

Antiviral therapies for viral URIs to date remain limited, yet physicians and health care providers are left with the professional duty to intervene in their patient's illness in some manner some prescribe therapies designed to mitigate the symptoms of viral URI since there is generally no therapeutic cure. Many of the medicines in this class can have side effects, complications, and undesirable medication interactions that prescribers and patients must be careful to avoid. Given their limited options, providers may feel pressured to prescribe antibiotics for viral upper respiratory infections when they are not necessary, or when there is a question if the infection is viral or bacterial, as it better to be safe than sorry. This practice, however, is leading to a situation of great concern in the health community. Indeed, it is now estimated that without behavioral changes to, and significantly reducing, antibiotic use, global death from drug resistant infections will surpass cancer-caused death by 2050 (see O'Neill J., (2014) Review on Antimicrobial Resistance: Tackling drug-resistant infections globally. http://amr-review.org/).

Current management of URIs revolves primarily around symptom reduction such as Tylenol for fever or Sudafed for nasal congestion, but these commonplace medications are ineffective in treating or reduce the duration of illness. Echinacea, vitamin C, and zinc are also used to either treat or reduce the symptoms of URis, generally independently. Such products include herbal medicinal Echinacea preparations like Echinilin®, Emergen-C® (a vitamin C-based product), and Cold-Eeze® (a zinc-based product). These products are claimed to have beneficial effect, and some studies appear to support those claims. However, each is primarily based upon a single ingredient (e.g., Echinacea, vitamin C or zinc), and is intended for use in a same-dosage regimen.

Echinacea is a coneflower native to the Western Hemisphere. Historically, Native American Plains Indians and European migrants used Echinacea as antimicrobial agent for skin wounds and upper respiratory infections. Echinacea, like many herbs, fell out of favor with the advent of modern antibiotics, particularly in the United States. However, it is now generally recognized that Echinacea supports the immune system's ability to clear infections by promoting T-cell activity, the cell type critical to cell-mediated immunity. As reported in the literature, Echinacea also stimulates interferon molecules in the immune system characterized by their antiviral properties. See McCann D. A., Solco A., Liu Y., Macaluso F., Murphy P. A., Kohut M. L., Senchina D. S. (2007); Cytokine- and interferon-modulating properties of Echinacea spp. root tinctures stored at −20 degrees C. for 2 years. Journal of Interferon Cytokine Research, May; 27(5):425-36; and. Senchina D. S., Martin A. E., Buss J. E., Kohut M. L. (2010); Effects of Echinacea extracts on macrophage antiviral activities. Phytotherapy Research, June:24(6):810-6. doi: 10.1002/ptr.2991.

As several reported studies have indicated, zinc is an essential trace metal with a wide range of critical functions in the human body. These include processes fundamental to life such as metabolism, gene expression, and enzyme function. As such, zinc is involved in virtually every aspect of our immune system's function, including the production, maturation, and function of white blood cells. See, Rink L., Gabriel P. (2000). Zinc and the immune system. Proceedings of the Nutrition Society, November; 59(4):541-52.; Shankar A. H., Prasad A. S. (1998). Zinc and immune function: the biological basis of altered resistance to infection. American Journal of Clinical Nutrition, August; 68(2 Suppl):447S-463S; and Wintergerst E. S., Maggini S., Hornig D. H. (2006) Immune-enhancing role of vitamin C and zinc and effect on clinical conditions. Annals of Nutrition & Metabolism, 50(2):85-94. Epub 2005 Dec. 21.

Vitamin C is a water-soluble vitamin critical to a wide range of metabolic reactions in the body, including the formation of collagen in bones, cartilage, muscle, and blood vessels, as well as the absorption of iron. Vitamin C is also found in high concentrations in cells of the immune system and is consumed during infections. It is thought that vitamin C is involved in the modulation of several immune cells that are involved in the clearance of viruses and bacteria as well as the propagation of cytokines that support the mounting of immune responses to infections. See, Wintergerst E. S., Maggini S., Hornig D. H. (2006), supra.

Advances in nanotechnology have created the opportunity to create novel methods to enhance the enteric uptake of immunomodulatory nutrients that support the immune system. These systems bridge the gap between hydrophilic and lipophilic interfaces to enhance absorption and targeting to ultimately improve efficacy of these agents.

Normally, during digestion, the gallbladder releases bile salts. Bile salts function as emulsifying agents allowing the small intestine to improve absorption of fatty or lipophilic substances. However, these bile salts are not released in great concentrations without the presence of a significant amount of chyme containing partially digested food transiting from the stomach. This release of bile salts is at times further reduced from disease states, nutritional deficiencies, and surgeries such as liver surgery or gallbladder removal.

The development of nanoparticles for therapeutic biomedical applications has allowed for the creation of particles containing therapeutic immunomodulators for URI treatment by creating a lipophilic interface that improves gut absorption of the medication in the presence or absence of bile salts and chyme. This allows for the effective targeted delivery of immunomodulatory preparations. Nanoparticles can be used to create a lipophilic interface to improve Echinacea, vitamin C, and zinc absorption while also providing a hydrophilic surface that allows the composition to be placed in hydrophilic solutions at the time of delivery while still maintaining enhanced absorption for optimizing therapeutic benefit. Honey can also be used as a stabilizing binder of lipophilic and hydrophilic compositions, while also having both antiviral and antibacterial properties.

A preferred embodiment of this invention utilizes a vitamin and mineral-added herbal blend composition in a nanoparticle delivery system packaged in a dose schedule method that supports the immune system's clearance of infections while allowing for reducing inappropriate antibiotic use.

### SUMMARY OF THE INVENTION

A composition of Echinacea, vitamin C, and zinc in a nanoparticle packaged for oral consumption in a varied dose schedule method of consumption that supports the immune system's clearance of infections, thereby allowing for reduced antibiotic use. The composition is composed of an Echinacea capsule, preferably made of vegetable cellulose and honey with the Echinacea particles contained within a liposome and a zinc-vitamin C capsule, preferably made of vegetable cellulose and honey with the zinc-vitamin C particles contained within a Janus nanoparticle for increased zinc and vitamin C absorption. Because of the liposome nanoparticle functioning similar to the physiological emulsifying agent bile, the absorption and therapeutic benefit of the composition is enhanced without the specific need or bile or chyme.

A single dose is composed of one Echinacea capsule and one zinc-vitamin C capsule. The use of Echinacea, zinc, and vitamin C in colds and flu, in the oral composition, amounts and dose schedule set forth herein optimize immune system support. Doses are scheduled with higher frequency at the initial onset of URI symptoms and are subsequently tapered off, preferably within 5 to 7 days.

### BRIEF DESCRIPTION OF THE DRAWING

FIG. 1 shows the preferred dosage method for one embodiment of the invention.

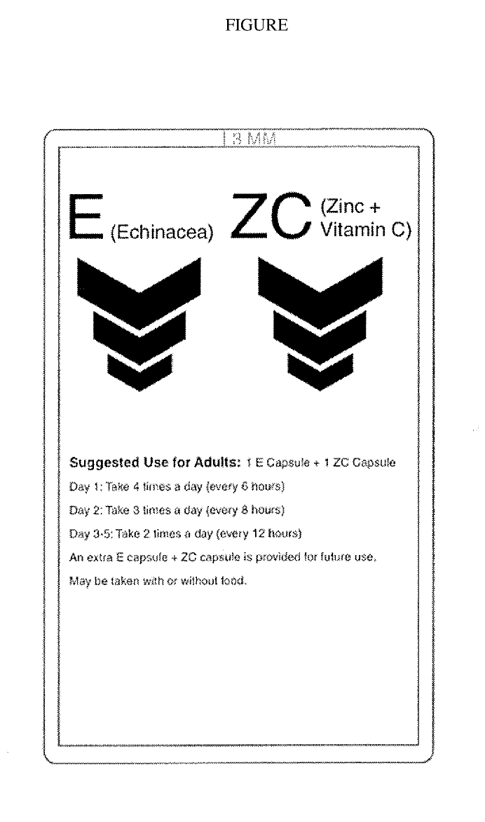

### DETAILED DESCRIPTION AND A PREFERRED EMBODIMENT

A preferred embodiment for the combination of ingredients and delivery is comprised of two capsules as a single dose taken in a dose schedule intended to maximize support of the patient's immune system. The first capsule contains Echinacea. The Echinacea dose range is 50 mg to 1000 mg. In a preferred embodiment, the Echinacea capsule dose is 550 mg of powdered Echinacea purpurea from the flower's root, seed, and aerial portions. The second capsule contains zinc and vitamin C. The dose range of the capsule is 3 mg to 150 mg of elemental zinc and 25 mg to 2000 mg of vitamin C. In a preferred embodiment, zinc is delivered in the form of zinc acetate powder at a dose of 25 mg of elemental zinc and the vitamin C is delivered in the form of powder at a dose of 250 mg.

The two capsules containing the Echinacea and the combined zinc and vitamin C dose can be composed of any delivery vehicle that can effectively deliver all of the components effectively into the bloodstream, including nanoparticles, such as a Janus nanoparticle, or honey capsule. In a preferred embodiment, the capsule is made of vegetable cellulose. Either capsule also contains an emulsifying agent that improves enteric absorption of capsule contents, such as a liposome, dendrimer, block co-polymer, or soy lecithin at a dose range of 100 mg to 500 mg. In a preferred embodiment, the combined zinc and vitamin C is delivered via liposome as the emulsifying agent inside the capsule.

The dose schedule is optimally initiated within 48 hours of the onset of URI symptoms. Doses are taken as often as every two hours and for as long as 14 days. In a preferred embodiment, doses are scheduled with higher frequency at the initial onset of URI symptoms and subsequently tapered off over 5 days. On day 1, a single dose is taken every 6 hours, or 4 times a day. On day 2, a single dose is taken every 8 hours, or 3 times a day. On days 3 through 5, a single dose is taken every 12 hours, or 2 times a day. In a preferred embodiment, an extra single dose is provided to take immediately the next time URI symptoms begin to develop. By initiating the dose schedule at the earliest sign of localized symptoms, a generalized upper respiratory infection has the highest chance of being prevented.

What is claimed is:

1. A composition and method of delivery comprising:
a. a first orally administered capsule containing between 50 mg and 1000 mg of powdered Echinacea purpurea from the flower's root, seed, and aerial portions;
b. a second orally administered capsule containing elemental zinc and vitamin C, with the range of zinc being between 3 mg and 150 mg, and the range of vitamin C being between 25 mg and 2000 mg;
c. a single dosage being one of said first capsules and one of said second capsules, taken at the same time;
d. said single dosage being take at least every 6 hours during the first 24 hours of consumption, at least every 8 hours during the second 24 hours of consumption, and at least every 12 hours during the third, fourth and fifth 24 hours of consumption; and
e. said capsules are of a vegetable cellulose and honey, and said zinc and vitamin C particles are contained with a nanoparticle that is conducive to increased absorption of the zinc and vitamin C.

2. The invention of claim 1 in which the dosage per said first capsule of Echinacea is between 450 and 650 mg; the dosage per said second capsule of zinc is between 20 and 30 mg; and the dosage per said second capsule of vitamin C is between 200 and 250 mg.
The Echinacea dose range is 50 mg to 1000 mg. In a preferred embodiment, the Echinacea capsule dose is 550 mg of powdered Echinacea purpurea from the flower's root, seed, and aerial portions. The second capsule contains zinc and vitamin C. The dose range of the capsule is 3 mg to 150 mg of elemental zinc and 25 mg to 2000 mg of vitamin C. In a preferred embodiment, zinc is delivered in the form of zinc acetate powder at a dose of 25 mg of elemental zinc and the vitamin C is delivered in the form of powder at a dose of 250 mg.
